(19) United States
(12) Patent Application Publication
(10) Pub. No.: US 2017/0313759 A1
(43) Pub. Date: Nov. 2, 2017

(54) NOVEL CHIMERIC ANTIGEN RECEPTORS

(71) Applicant: GLAXOSMITHKLINE INTELLECTUAL PROPERTY DEVELOPMENT LIMITED, Brentford (GB)

(72) Inventor: Thil Dinuk BATUWANGALA, Stevenage (GB)

(21) Appl. No.: 15/291,226

(22) Filed: Oct. 12, 2016

(30) Foreign Application Priority Data
Oct. 14, 2015 (GB) 1518136.5

(51) Int. Cl. C07K 14/73; C07K 14/725; C07K 16/30; A61K 35/17; C12N 5/0783; C07K 14/705; A61K 35/12

(52) U.S. Cl. CPC C07K 14/70514; C12N 5/0636; C07K 14/7051; C07K 14/70521; C07K 16/30; A61K 35/17; A61K 2035/124; C07K 2317/92; C07K 2319/02; C07K 2319/03; C12N 2510/00; C07K 2317/622

(57) ABSTRACT

The invention relates to chimeric antigen receptor (CAR) scaffolds comprising: a target binding domain; a spacer region; a transmembrane domain; and an intracellular effector domain, wherein the spacer region comprises at least one, or multiples of, domains 2, 3 or 4 or a combination thereof of a CD4 molecule. The invention also relates to polynucleotides and expression vectors encoding said CAR scaffold and immunomodulatory cells comprising said CAR scaffold. The invention also relates to methods of engineering an immunomodulatory cell to comprise said CAR scaffold

### CROSS REFERENCE TO RELATED APPLICATIONS

This application claims the benefit of U.K. Provisional Application No. GB 1518136.5, filed 14 Oct. 2015.

### FIELD OF THE INVENTION

The invention relates to chimeric antigen receptors comprising a spacer region from a CD4 molecule. The present invention also relates to polynucleotides and vectors encoding said CAR and immunomodulatory cells expressing said CAR at their surface. The present invention also relates to methods for engineering immune cells expressing said CAR at their surface.

### BACKGROUND TO THE INVENTION

T cells of the immune system recognize and interact with specific antigens through T cell receptors (TCRs) which, upon recognition or binding with such antigens, causes activation of the cell. TCRs are expressed on the T cell surface and comprise highly variable protein chains (such as alpha (α) and beta (β) chains) which are expressed as part of a complex with CD3 chain molecules. The CD3 chain molecules have an invariant structure and, in particular, the CD3zeta (CD3ζ) chain is responsible for intracellular signalling upon TCR:antigen binding. The TCRs recognise antigenic peptides that are presented to it by the proteins of the major histocompatibility complex (MHC) which are expressed on the surface of antigen presenting cells and other T cell targets. In natural CD8⁺ T cell activation, antigenic peptides presented by MHC Class I on antigen presenting cells are recognised by the TCR and the TCR:peptide:MHC complex is formed. This forms intercellular membrane contact regions that are defined in width by the physical dimensions of the TCR:peptide:MHC complex. Inhibitory signalling receptors that are too large to fit in this space are excluded allowing triggering of the TCR/CD3 signals to activate cell killing (Choudhuri et al. (2005) Nature 436 (7050):578-582).

Chimeric antigen receptors (CARs) have been developed as artificial TCRs to generate novel specificities in T cells without the need to bind to MHC-antigenic peptide complexes. These synthetic receptors contain a target binding domain that is associated with one or more signalling domains via a flexible linker in a single fusion molecule. The target binding domain is used to target the T cell to specific targets on the surface of pathologic cells and the signalling domains contain molecular machinery for T cell activation and proliferation. The flexible linker which passes through the T cell membrane (i.e. forming a transmembrane domain) allows for cell membrane display of the target binding domain of the CAR. CARs have successfully allowed T cells to be redirected against antigens expressed at the surface of tumour cells from various malignancies including lymphomas and solid tumours (Jena et al. (2010) Blood, 116(7):1035-44).

The development of CARs has comprised three generations so far. The first generation CARs comprised target binding domains attached to a signalling domain derived from the cytoplasmic region of the CD3zeta or the Fc receptor gamma chains. First generation CARs were shown to successfully redirect T cells to the selected target, however, they failed to provide prolonged expansion and antitumor activity in vivo. The second and third generation CARs have focussed on enhancing modified T cell survival and increasing proliferation by including co-stimulatory molecules, such as CD28, OX-40 (CD134) and 4-1BB (CD137).

T cells bearing CARs could be used to eliminate pathologic cells in a disease setting. One clinical aim would be to transform patient cells with recombinant DNA containing an expression construct for the CAR via a vector (e.g. a lentiviral vector) following aphaeresis and T cell isolation. Following expansion of the T cells they are re-introduced into the patient with the aim of targeting and killing the pathologic target cells.

However, there is still a need in the art to develop the construction of CARs to provide improved characteristics, such as enhanced binding properties. It is therefore an object of the present invention to provide CARs with improved characteristics.

### SUMMARY OF THE INVENTION

According to a first aspect of the invention there is provided a chimeric antigen receptor (CAR) comprising:

a target binding domain;

a spacer region;

a transmembrane domain; and

an intracellular effector domain,

wherein the spacer region comprises at least one, or multiples of, domains 2, 3 or 4 or a combination thereof of a CD4 molecule.

According to a further aspect of the invention, there is provided a polynucleotide encoding the chimeric antigen receptor described herein.

According to a further aspect of the invention, there is provided an expression vector comprising the polynucleotide described herein.

According to a further aspect of the invention, there is provided an immunomodulatory cell comprising the chimeric antigen receptor described herein.

According to a further aspect of the invention, there is provided the immunomodulatory cell described herein for use in therapy.

According to a further aspect of the invention, there is provided a method of engineering an immunomodulatory cell, comprising:

(a) providing an immunomodulatory cell;

(b) introducing the expression vector described herein into said immunomodulatory cell; and

(c) expressing said expression vector in the immunomodulatory cell.

According to a further aspect of the invention, there is provided an engineered immunomodulatory cell comprising a chimeric antigen receptor (CAR) which binds to a protein on a target cell, wherein said CAR comprises:

a target binding domain,

a spacer domain which comprises at least one, or multiples of, domains 2, 3 or 4 or a combination thereof of a CD4 molecule,

a transmembrane domain and

an intracellular effector domain,

wherein the length of the spacer domain is such that the distance between the cell membranes of the target cell and engineered immunomodulatory cell creates an immune synapse.

### BRIEF DESCRIPTION OF THE FIGURES

FIG. 1: Modelling of CAR with CD4 spacers. Structures are to scale and rendered as low-resolution globular surfaces. Spheres on scFV and CD4 domains show termini of protein chains where polypeptide chains could fuse. 14 nm is the calculated distance between T-cell membrane (solid black line) and target cell membrane (top dotted grey line). In the case of BCMA, based on this modelling it is predicted that a type-2 CAR spacer gives optimal spacing for binding.

FIG. 2: Cytotoxicity of transduced αBCMA CAR T-cells specific to target expressing cells. A) Gating strategy: gates were drawn around T-cells and target cells and counts used to determine ratios over control. B) Percentage (%) cytotoxicity of CAR T-cells as assessed by flow cytometry. Co-cultured transduced T-cells and target cells were incubated for 24 hours at an effector to target ratio of 1:1. A baseline % cytotoxicity is observed for negative target cells comparable to untransduced (UT) T-cells. All CD4 spacer variants show significant cytotoxic activity over background comparable to the CD8 comparator spacer. Nomenclature to FIG. 1: ‘No spacer’ is type-0; ‘short spacer’ is type-1; ‘intermediate spacer’ is type-2; ‘long spacer’ is type-3.

FIG. 3: Target specific cytokine expression after incubation with αBCMA CAR T-cells. A) Gating strategy to identify cytokine producing cells. B) IFN-γ and IL-2 specific staining, respectively, of target cells over control. Variable levels of cytokine production are possibly dependent on spacer length. Colour coding of bars and nomenclature of constructs as in FIG. 2.

### DETAILED DESCRIPTION OF THE INVENTION

### Definitions

Unless defined otherwise, all technical and scientific terms used herein have the same meaning as is commonly understood by one of skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2^{nd} ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference in their entirety) and chemical methods. All patents and publications referred to herein are incorporated by reference in their entirety.

The term “comprising” encompasses “including” or “consisting” e.g. a composition “comprising” X may consist exclusively of X or may include something additional e.g. X+Y.

The term “consisting essentially of” limits the scope of the feature to the specified materials or steps and those that do not materially affect the basic characteristic(s) of the claimed feature.

The term “consisting of” excludes the presence of any additional component(s).

The term “about” as used herein when referring to a measurable value such as an amount, a temporal duration, and the like, is meant to encompass variations of ±20% or ±10%, including ±5%, ±1%, and ±0.1% from the specified value.

The term “chimeric antigen receptors” (“CARs”) as used herein, refers to an engineered receptor which consists of an extracellular target binding domain (which is usually derived from a monoclonal antibody or fragment thereof), a spacer region, a transmembrane region, and one or more intracellular effector domains. CARs have also been referred to as chimeric T cell receptors or chimeric immunoreceptors (CIRs). CARs are genetically introduced into hematopoietic cells, such as T cells, to redirect specificity for a desired cell-surface antigen.

The term “target binding domain” as used herein is defined as an oligo- or polypeptide that is capable of binding a specific target, such as an antigen or ligand. In particular, the target may be a cell surface molecule. For example, the target binding domain may be chosen to recognise a target that acts as a cell surface marker on pathogenic cells, including pathogenic human cells, associated with a particular disease state.

The term “spacer region” as used herein, refers to an oligo- or polypeptide that functions to link the transmembrane domain to the target binding domain. This region may also be referred to as a “hinge region” or “stalk region”. As explained in more detail herein, the size of the spacer can be varied depending on the position of the target epitope in order to maintain a set distance (e.g. 14 nm) upon CAR:target binding.

The term “domain” refers to a folded protein structure which retains its tertiary structure independent of the rest of the protein. Generally domains are responsible for discrete functional properties of proteins and in many cases may be added, removed or transferred to other proteins without loss of function of the remainder of the protein and/or of the domain.

The term “transmembrane domain” as used herein refers to the part of the CAR molecule which traverses the cell membrane.

The term “intracellular effector domain” (also referred to as the “signalling domain”) as used herein refers to the domain in the CAR which is responsible for intracellular signalling following the binding of the target binding domain to the target. The intracellular effector domain is responsible for the activation of at least one of the normal effector functions of the immune cell in which the CAR is expressed. For example, the effector function of a T cell can be a cytolytic activity or helper activity including the secretion of cytokines.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., VH, VHH, VL, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing.

The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb™” may be considered the same as a “single variable domain”. A single variable domain may be a human single variable domain, but also includes single variable domains from other species such as rodent (for example, as disclosed in WO 00/29004), nurse shark and Camelid VHH dAbs™ Camelid VHH are immunoglobulin single variable domain polypeptides that are derived from camelid species including bactrian and dromedary camels, llamas, vicugnas, alpacas, and guanacos, which produce heavy chain antibodies naturally devoid of light chains. Such VHH domains may be humanised according to standard techniques available in the art, and such domains are considered to be “single variable domains”. As used herein VH includes camelid VHH domains.

“Affinity” is the strength of binding of one molecule, e.g. the target binding protein of the CAR molecule of the invention, to another, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. BIACORE™ analysis).

The term “epitope” as used herein refers to that portion of the antigen that makes contact with a particular binding domain, e.g. the target binding domain of the CAR molecule. An epitope may be linear or conformational/discontinuous. A conformational or discontinuous epitope comprises amino acid residues that are separated by other sequences, i.e. not in a continuous sequence in the antigen's primary sequence. Although the residues may be from different regions of the peptide chain, they are in close proximity in the three dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography.

Sequence identity as used herein is the degree of relatedness between two or more amino acid sequences, or two or more nucleic acid sequences, as determined by comparing the sequences. The comparison of sequences and determination of sequence identity may be accomplished using a mathematical algorithm; those skilled in the art will be aware of computer programs available to align two sequences and determine the percent identity between them. The skilled person will appreciate that different algorithms may yield slightly different results.

Thus the “percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

Similarly, the “percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The terms “individual”, “subject” and “patient” are used herein interchangeably. In one embodiment, the subject is a mammal, such as a primate, for example a marmoset or monkey, or a human. In a further embodiment, the subject is a human.

The CAR described herein may also be used in methods of treatment of a subject in need thereof. Treatment can be therapeutic, prophylactic or preventative. Treatment encompasses alleviation, reduction, or prevention of at least one aspect or symptom of a disease and encompasses prevention or cure of the diseases described herein.

The CAR described herein is used in an effective amount for therapeutic, prophylactic or preventative treatment. A “therapeutically effective amount” of the antigen binding protein described herein is an amount effective to ameliorate or reduce one or more symptoms of, or to prevent or cure, the disease. The “therapeutically effective amount” also refers to the amount of the antigen binding protein described herein that will elicit the biological or medical response of a tissue, system, or subject that is being sought by a researcher, veterinarian, medical doctor or other clinician. The term “therapeutically effective amount” includes that amount of an antigen binding protein that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass, e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenström's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa associated lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenström's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), plasma cell leukemia, and primary amyloidosis (AL). Hematopoietic cancers may also include cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

The term “anti-tumor effect” as used herein, refers to a biological effect which can be manifested by various means, including but not limited to, a decrease in tumor volume, a decrease in the number of tumor cells, a decrease in the number of metastases, an increase in life expectancy, a decrease in tumor cell proliferation, a decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

### Chimeric Antigen Receptors

The present inventors have developed a CAR scaffold with improved binding properties by introducing a spacer region comprising the domains of a CD4 molecule. In natural CD8⁺ T cell activation, the TCR:peptide:MHC complex which is formed on antigen binding creates intercellular membrane contact regions that are defined in width by the physical dimensions of the TCR:peptide:MHC complex. Inhibitory signalling receptors that are too large to fit in this space are excluded allowing triggering of the TCR:CD3 signals to activate cell killing (see Choudhuri et al. (2005) Nature 436(7050):578-582). The present inventors have developed a method of designing CARs which takes into account this phenomenon of exclusion of inhibitory receptors by using spacer regions which are designed to mimic the dimensions of the TCR:peptide:MHC complex. For example, if the target epitope for the scFv is close to the target cell membrane then a larger spacer would be required for the scFv to reach it while maintaining the set distance between membranes. Dimensions of the TCR:peptide:MHC complex are such that the distance between membranes of opposing cells would be approximately 14 nm/14 Å (see Wild et al. (1999) J. Exp. Med., 190(1):31-41, Garboczi et al. (1996) Nature, 384:134-141, Garcia et al. (1998) Science, 279:1166-1172)

Previously, spacers have been investigated using IgG Fc domains (e.g. see WO2014/031687 and Guest et al. (2005) J. Immunother., 28(3):203-211, herein incorporated by reference). The problem with IgG Fc domains is that they naturally dimerise and form interactions with other molecules. The present inventors recognized that a preferred CAR spacer domain would be as inert as possible so that it does not affect the binding ability of the target binding domain of the CAR scaffold. The present invention utilizes CD4 domains 2, 3 and/or 4 as a spacer, as these domains are inert and do not form dimerising interactions with other molecules.

Therefore, according to a first aspect of the invention there is provided a chimeric antigen receptor (CAR) comprising:

a target binding domain;

a spacer region;

a transmembrane domain; and

an intracellular effector domain,

wherein the spacer region comprises at least one, or multiples of, domains 2, 3 or 4 or a combination thereof of a CD4 molecule.

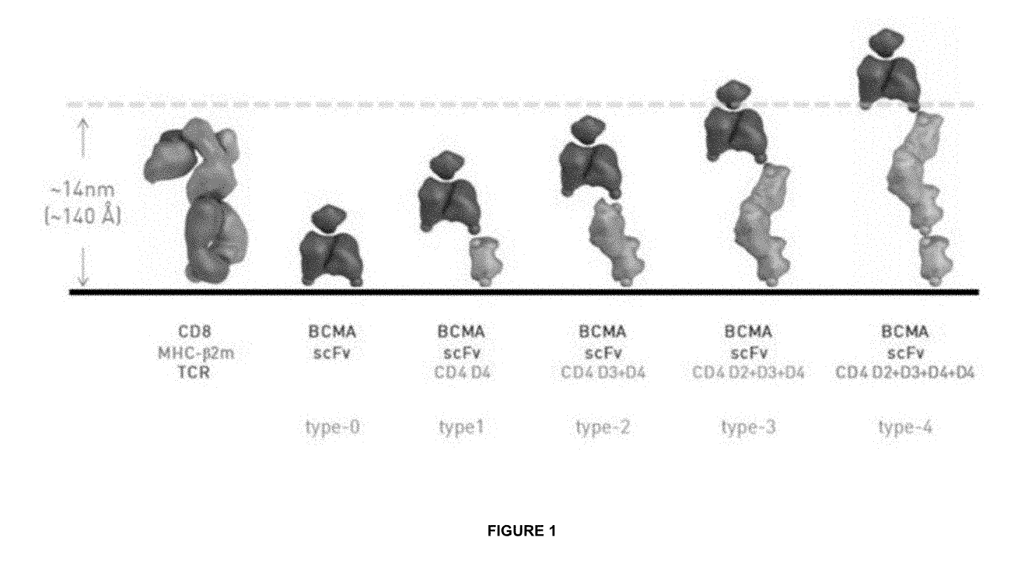

The epitope of an antigen may be positioned proximal (i.e. near) to the target cell membrane or distal (i.e. far) from the target cell membrane. Another factor which affects the size of spacer to be chosen is the size of the target molecule. As depicted in FIG. 1, one example is the antigen BCMA (B-cell maturation antigen). This is a small antigen with the target epitope distal from the target cell membrane. Based on the modelling in FIG. 1, the present inventors predicted that the most effective spacer to use in a BCMA-specific CAR is type-2 (i.e. a spacer comprising domains 3 and 4 of a CD4 molecule) because this results in a 14 nm distance between the target and T cell membranes upon CAR:antigen binding.

Thus, according to the present invention, the size of the spacer is selected based upon the epitope position and/or size of the target antigen. For example, the CEA antigen is relatively large, but the epitope is positioned distal from the target cell membrane, therefore only a short spacer would be needed to improve CAR binding properties. In an alternative example, the NCAM (natural cell adhesion molecule) antigen is also relatively large, but the epitope is positioned proximal to the target cell membrane, therefore a large spacer would be needed to improve CAR binding properties. The size of the spacer selected for use in the CAR can therefore be decided when the target is selected based on the size of the target and position of the epitope. Methods of epitope mapping, in order to determine the position of a target epitope, are well known in the art, such as X-ray co-crystallography, array-based oligopeptide scanning (or pepscan analysis) and site directed mutagenesis.

The term “CD4” as used herein, refers to a Cluster of Differentiation 4 molecule which is a member of the immunoglobulin superfamily. CD4 is a co-receptor that assists in the TCR:peptide:MHC Class II interaction and has a rigid rod-like structure. It has four immunoglobulin domains (domains 1 to 4) that are exposed on the extracellular surface of the cell. CD4 binds MHC Class II via domain 1, whilst domains 2-4 act as a scaffold (Yin et al. (2012) PNAS, 109(14):5405-5410). The amino acid sequence of CD4 is described in more detail on UniProt, ID number P01730. The term “domain” refers to a folded protein structure which retains its tertiary structure independent of the rest of the protein. Generally domains are responsible for discrete functional properties of proteins and in many cases may be added, removed or transferred to other proteins without loss of function of the remainder of the protein and/or of the domain.

In one embodiment, the spacer region comprises or consists of domain 4 of a CD4 molecule. The spacer region may comprise at least one copy of domain 4 of a CD4 molecule. For example, in a further embodiment, the spacer region comprises or consists of multiple copies of domain 4 of a CD4 molecule (e.g. 2, 3 or 4 copies).

In one embodiment, the spacer region comprises or consists of domain 3 of a CD4 molecule. The spacer region may comprise at least one copy of domain 3 of a CD4 molecule. For example, in a further embodiment, the spacer region comprises or consists of multiple copies of domain 3 of a CD4 molecule (e.g. 2, 3 or 4 copies).

In one embodiment, the spacer region comprises or consists of domain 2 of a CD4 molecule. The spacer region may comprise at least one copy of domain 2 of a CD4 molecule. For example, in a further embodiment, the spacer region comprises or consists of multiple copies of domain 2 of a CD4 molecule (e.g. 2, 3 or 4 copies).

In one embodiment, the spacer region comprises or consists of domains 3 and 4 or combinations thereof of a CD4 molecule. For example, the spacer region may comprise or consist of one copy of each domain 3 and domain 4 of a CD4 molecule, or the spacer region may comprise or consist of one copy of domain 3 and two copies of domain 4 of a CD4 molecule, or vice versa.

In one embodiment, the spacer region comprises or consists of domains 2, 3 and 4 or combinations thereof of a CD4 molecule. In a further embodiment, the spacer region comprises or consists of domains 2 and 3 and two copies of domain 4 of a CD4 molecule.

In one embodiment, the spacer domain comprises or consists of an amino acid sequence with at least 70%, preferably at least 80%, more preferably at least 90%, 95% 97% or 99% sequence identity with an amino acid sequence selected from the group consisting of: SEQ ID NOs 2, 3 and 4. In a further embodiment, the spacer region comprises or consists of an amino acid sequence selected from the group consisting of: SEQ ID NOs 2, 3 and 4.

The advantage of using CD4 domains is that they are easy to manipulate in order to create a spacer of the desired size depending on the epitope position and/or size of the target. Furthermore, domains 2, 3 and 4 of CD4 are relatively inert which makes them ideal for use as a spacer because they would not affect the target binding domain of the CAR molecule. The flexibility or rigidity of the spacer can also be tailored by using domains 2, 3 and 4 of CD4. Naturally, the connection between domains 2 and 3 of CD4 is more flexible than the connection between domains 3 and 4. Therefore, if a more flexible CAR scaffold is required, then domains 2 and 3 of CD4 can be used, whereas if the CAR scaffold is required to be more rigid then domains 3 and 4 of CD4 can be used.

The boundaries of the CD4 domains are disclosed in more detail herein, however it will be understood by a person skilled in the art that the CD4 domains may be as defined by any domain databases, such as Uniprot or Interpro.

The CD4 molecule also contains domain 1 which binds MHC Class II, therefore it will be understood that this domain is not suitable for use as a spacer according to the present invention because the CAR molecule is not required to interact with an MHC molecule. Therefore, in one embodiment, the spacer region does not comprise domain 1 of a CD4 molecule. In one embodiment, domain 1 of a CD4 molecule starts at any one of amino acids 20 to 31 (i.e. amino acid 21, 22, 23, 24, 25, 26, 27, 28, 29, 30 or 31) of SEQ ID NO: 1 and ends at any one of amino acids 116 to 125 (i.e. amino acid 116, 117, 118, 119, 120, 121, 122, 123, 124 or 125) of SEQ ID NO: 1. In one embodiment, domain 1 of a CD4 molecule comprises amino acids 31 to 116 of SEQ ID NO: 1. In a further embodiment, domain 1 of a CD4 molecule comprises amino acids 26 to 125 of SEQ ID NO: 1. In an alternative embodiment, domain 1 of a CD4 molecule comprises amino acids 20 to 120 of SEQ ID NO: 1.

In one embodiment, domain 2 of a CD4 molecule starts at any one of amino acids 123 to 126 (i.e. amino acid 123, 124, 125 or 126) of SEQ ID NO: 1 and ends at any one of amino acids 197 to 203 (i.e. amino acid 197, 198, 199, 200, 201, 202 or 203) of SEQ ID NO: 1. In a further embodiment, domain 2 of a CD4 molecule comprises amino acids 126 to 197 of SEQ ID NO: 1. In a yet further embodiment, domain 2 of a CD4 molecule comprises amino acids 123 to 201 of SEQ ID NO: 1. In an alternative embodiment, domain 2 of a CD4 molecule comprises amino acids 126 to 203 of SEQ ID NO: 1. In another alternative embodiment, domain 2 of a CD4 molecule comprises amino acids 125 to 203 of SEQ ID NO: 1. In one embodiment, domain 3 of a CD4 molecule starts at any one of amino acids 202 to 208 (i.e. amino acids 202, 203, 204, 205, 206, 207 or 208) of SEQ ID NO: 1 and ends at amino acid 316 or 317 of SEQ ID NO: 1. In a further embodiment, domain 3 of a CD4 molecule comprises amino acids 208 to 316 of SEQ ID NO: 1. In a yet further embodiment, domain 3 of a CD4 molecule comprises amino acids 202 to 317 of SEQ ID NO: 1. In an alternative embodiment, domain 3 of a CD4 molecule comprises amino acids 204 to 316 of SEQ ID NO: 1.

In one embodiment, domain 4 of a CD4 molecule starts at any one of amino acids 315 to 318 (i.e. amino acids 315, 316, 317 or 318) of SEQ ID NO: 1 and ends at any one of amino acids 374 to 396 (e.g. 386 or 388) of SEQ ID NO: 1. In a further embodiment, domain 4 of a CD4 molecule comprises amino acids 318 to 374 of SEQ ID NO: 1. In a yet further embodiment, domain 4 of a CD4 molecule comprises amino acids 318 to 396 of SEQ ID NO: 1. In an alternative embodiment, domain 4 of the CD4 molecule comprises amino acids 318 to 388 of SEQ ID NO: 1. In another alternative embodiment, domain 4 of a CD4 molecule comprises amino acids 315 to 386 of SEQ ID NO: 1.

It will be understood that the sequences described herein may further comprise sequences to aid with cloning and expression of the CD4 domains. For example, amino acid sequences such as “FGL”, “SVRS” or “LA” can be added to the synthesised domain to aid with cloning. Furthermore, the sequences of the domains as defined herein are based upon data available on the UniProt protein database, however it would be understood that the domain boundaries are not restricted to only those as defined on this database.

The target binding domain binds to a target, wherein the target is a tumour specific molecule, viral molecule, or any other molecule expressed on a target cell population that is suitable to mediate recognition and elimination by a lymphocyte. In one embodiment, the target binding domain comprises an antibody, an antigen binding fragment or a ligand. In one embodiment, the target binding domain comprises an antibody or fragment thereof. In one embodiment, the target binding domain is a ligand. In an alternative embodiment, the target binding domain is an antigen binding fragment. In a further embodiment, the antigen binding fragment is a single chain variable fragment (scFv) or a dAb™. In a yet further embodiment, said scFv comprises the light (VL) and the heavy (VH) variable fragment of a target antigen specific monoclonal antibody joined by a flexible linker. In one embodiment, the target binding domain may bind to more than one target, for example two different targets. Such a target binding domain may be derived from a bispecific single chain antibody. For example, Blinatumomab (also known as AMG 103 or MT103) is a recombinant CD19 and CD3 bispecific scFv antibody consisting of four immunoglobulin variable domains assembled into a single polypeptide chain. Two of the variable domains form the binding site for CD19 which is a cell surface antigen expressed on most normal and malignant B cells. The other two variable domains form the binding site for CD3 which is part of the T cell-receptor complex on T cells. These variable domains may be arranged in the CAR in tandem, i.e. two single chain antibody variable fragments (scFv) tethered to a spacer, and transmembrane and signaling domains. The four variable domains can be arranged in any particular order within the CAR molecule (e.g. VL(first target)-VH(first target)-VH(second target)-VL(second target) or VL(second target)-VH(second target)-VH(first target)-VL(first target) etc.).

In one embodiment, the target binding domain and/or spacer domain may comprise a multimerization domain(s), for example as described in WO2015/017214. This enables the signal transduction of the CAR to be controlled through the addition of external agents, such as a chemical drug, which acts a bridging factor between the multimerization domains. Therefore, in one embodiment, the target binding domain and/or spacer domain comprises (a) a first multimerization domain; and (b) a second multimerization domain; wherein a first bridging factor promotes the formation of a polypeptide complex with the bridging factor associated with and disposed between the first and second multimerization domains.

The target binding domain may bind a variety of cell surface antigens, but in one embodiment, the target binding domain binds to a tumour associated antigen. In a further embodiment, the tumor associated antigen is selected from: BCMA, CD19, HER2, prostate stem cell antigen (PSCA), prostate-specific membrane antigen (PSMA), carcinoembryonic antigen (CEA), cancer antigen-125, CA19-9, MUC-1, tyrosinase, CD34, CD45, CD117, protein melan-A, synaptophysis, CD22, CD27, CD30, CD70, ganglioside G2 (GD2), epidermal growth factor variant III (EGFRvIII), mesothelin, prostatic acid phosphatise (PAP), prostein, TARP, Trp-p8 or six transmembrane epithelial antigen of the prostate I (STEAP1). In a yet further embodiment, the tumour associated antigen is BCMA.

In one embodiment, the target binding domain has a binding affinity of less than about 500 nanomolar (nM), such as less than about 400 nM, 350 nM, 300 nM, 250 nM, 200 nM, 150 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 0.5 nM or 0.25 nM. In one embodiment, the target binding domain has a binding affinity of about 10 nM to about 0.25 nM. In a further embodiment, the target binding domain has a binding affinity of about 1 nM to about 0.5 nM (i.e. about 1000 pM to about 500 pM).

In one embodiment, the transmembrane domain can be derived either from a natural or from a synthetic source. In one embodiment, the transmembrane domain can be derived from any membrane-bound or transmembrane protein. Alternatively the transmembrane domain can be synthetic and can comprise predominantly hydrophobic residues such as leucine and valine.

For example, the transmembrane domain can be the transmembrane domain of CD proteins, such as CD4, CD8, CD3 or CD28, a subunit of the T cell receptor, such as α, β, γ or δ, a subunit of the IL-2 receptor (α chain), a submit of the Low-Affinity Nerve Growth Factor Receptor (LNGFR or p75) (β chain or γ chain), or a subunit chain of Fc receptors. In one embodiment, the transmembrane domain comprises the transmembrane domain of CD4, CD8 or CD28. In a further embodiment, the transmembrane domain comprises the transmembrane domain of CD4 or CD8 (e.g. the CD8 alpha chain, as described in NCBI Reference Sequence: NP_001139345.1, incorporated herein by reference). In a yet further embodiment, the transmembrane domain comprises the transmembrane domain of CD4. The advantage of this embodiment is that the CD4 transmembrane domain is joined to the CD4 spacer domains, therefore this avoids using an unnatural junction and the CAR molecule is easier to construct. This is particularly advantageous over the prior art which describes using IgG domains as the spacer because these domains would not normally be linked to a transmembrane domain therefore they are forced into an unnatural junction which may affect the ability of the CAR scaffold to bind to a target.

In one embodiment, the transmembrane domain of the CD4 molecule comprises amino acids 397 to 418 of SEQ ID NO: 1. In a further embodiment, the transmembrane domain of the CD4 molecule comprises a sequence which starts at any one of amino acids 375 to 397 (e.g. 389) of SEQ ID NO: 1 and ends at amino acid 418 of SEQ ID NO: 1.

In one embodiment, the transmembrane domain comprises an amino acid sequence with at least 70%, preferably at least 80%, more preferably at least 90%, 95 97% or 99% sequence identity with an amino acid sequence of SEQ ID NO: 5. In a further embodiment, the transmembrane region comprises an amino acid sequence of SEQ ID NO: 5.

Preferred examples of the effector domain for use in a CAR scaffold can be the cytoplasmic sequences of the natural T cell receptor and co-receptors that act in concert to initiate signal transduction following antigen binding, as well as any derivate or variant of these sequences and any synthetic sequence that has the same functional capability. Effector domains can be separated into two classes: those that initiate antigen-dependent primary activation, and those that act in an antigen-independent manner to provide a secondary or costimulatory signal. Primary activation effector domains can comprise signalling motifs which are known as immunoreceptor tyrosine-based activation motifs (ITAMs). ITAMs are well defined signalling motifs, commonly found in the intracytoplasmic tail of a variety of receptors, and serve as binding sites for syk/zap70 class tyrosine kinases. Examples of ITAMs used in the invention can include, as non limiting examples, those derived from CD3zeta, FcRgamma, FcRbeta, FcRepsilon, CD3gamma, CD3delta, CD3epsilon, CD5, CD22, CD79a, CD79b and CD66d. In one embodiment, the intracellular effector domain comprises a CD3zeta signalling domain (also known as CD247). Natural TCRs contain a CD3zeta signalling molecule, therefore the use of this effector domain is closest to the TCR construct which occurs in nature.

In one embodiment, the intracellular effector domain of the CAR comprises a CD3zeta signalling domain which has an amino acid sequence with at least 70%, preferably at least 80%, more preferably at least 90%, 95% 97% or 99% sequence identity with SEQ ID NO: 7. In a further embodiment, the intracellular effector domain of the CAR comprises a CD3zeta signalling domain which comprises an amino acid sequence of SEQ ID NO: 7.

As described herein, effector domains may also provide a secondary or costimulatory signal. T cells additionally comprise costimulatory molecules which bind to cognate costimulatory ligands on antigen presenting cells in order to enhance the T cell response, for example by increasing proliferation activation, differentiation and the like. Therefore, in one embodiment, the intracellular effector domain additionally comprises a costimulatory domain. In a further embodiment, the costimulatory domain comprises the intracellular domain of a costimulatory molecule, selected from CD28, CD27, 4-1BB (CD137), OX40 (CD134), ICOS (CD278), CD30, CD40, PD-1 (CD279), CD2, CD7, NKG2C (CD94), B7-H3 (CD276) or any combination thereof. In a yet further embodiment, the costimulatory domain comprises the intracellular domain of a costimulatory molecule, selected from CD28, CD27, 4-1BB, OX40, ICOS or any combination thereof.

In one embodiment, the intracellular effector domain additionally comprises a CD28 intracellular domain which has an amino acid sequence with at least 70%, preferably at least 80%, more preferably at least 90%, 95% 97% or 99% sequence identity with SEQ ID NO: 6. In a further embodiment, the intracellular effector domain additionally comprises a CD28 intracellular domain which comprises an amino acid sequence of SEQ ID NO: 6.

### Polynucleotides and Expression Vectors

According to a further aspect of the invention, there is provided a polynucleotide encoding the chimeric antigen receptor described herein.

The polynucleotide may be present in an expression cassette or expression vector (e.g. a plasmid for introduction into a bacterial host cell, or a viral vector such as a lentivirus for transfection of a mammalian host cell). Therefore, according to a further aspect of the invention, there is provided an expression vector comprising the polynucleotide described herein.

The term “vector” refers to a vehicle which is able to artificially carry foreign genetic material into another cell, where it can be replicated and/or expressed. In one embodiment, the expression vector is a retroviral vector. In a further embodiment, the retroviral vector is derived from, or selected from, a lentivirus, alpha-retrovirus, gamma-retrovirus or foamy-retrovirus, such as a lentivirus or gamma-retrovirus, in particular a lentivirus. In a further embodiment, the retroviral vector particle is a lentivirus selected from the group consisting of HIV-1, HIV-2, SIV, FIV, EIAV and Visna. Lentiviruses are able to infect non-dividing (i.e. quiescent) cells which makes them attractive vectors for gene therapy. In a yet further embodiment, the retroviral vector particle is HIV-1 or is derived from HIV-1. The genomic structure of some retroviruses may be found in the art. For example, details on HIV-1 may be found from the NCBI Genbank (Genome Accession No. AF033819). HIV-1 is one of the best understood retroviruses and is therefore often used as a viral vector.

### Immunomodulatory Cells

According to a further aspect of the invention, there is provided an immunomodulatory cell comprising the chimeric antigen receptor described herein. In one embodiment, the immunomodulatory cell may be a human immunomodulatory cell.

The term “immunomodulatory cell” refers to a cell of hematopoietic origin functionally involved in the modulation (e.g. the initiation and/or execution) of the innate and/or adaptive immune response. Said immunomodulatory cell according to the present invention can be derived from a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Said immunomodulatory cell can also be a dendritic cell, a killer dendritic cell, a mast cell, a NK-cell, a B-cell or a T-cell. The T-cell may be selected from the group consisting of inflammatory T-lymphocytes, cytotoxic T-lymphocytes, regulatory T-lymphocytes or helper T-lymphocytes, or a combination thereof. Therefore, in one embodiment, the immunomodulatory cell is derived from an inflammatory T-lymphocyte, cytotoxic T-lymphocyte, regulatory T-lymphocyte or helper T-lymphocyte. In another embodiment, said cell can be derived from the group consisting of CD4⁺ T-lymphocytes and CD8⁺ T-lymphocytes.

Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used. In another embodiment, said cell can be derived from a healthy donor or a diseased donor, such as a patient diagnosed with cancer or an infection. In another embodiment, said cell is part of a mixed population of cells which present different phenotypic characteristics.

It will be understood that the immunomodulatory cells may express the chimeric antigen receptor described herein transiently or stably/permanently (depending on the transfection method used and whether the polynucleotide encoding the chimeric antigen receptor has integrated into the immunomodulatory cell genome or not).

### Uses

According to a further aspect of the invention, there is provided a method of treatment of a patient in need thereof, comprising administering the immunomodulatory cell described herein to a human subject in need of such therapy.

In one embodiment, the therapy is adoptive cellular therapy. “Adoptive cellular therapy” (or “adoptive immunotherapy”) refers to the adoptive transfer of human T lymphocytes that are engineered by gene transfer to express CARs (such as the CARs of the present invention) specific for surface molecules expressed on target cells. This can be used to treat a range of diseases depending upon the target chosen, e.g. tumour specific antigens to treat cancer. Adoptive cellular therapy involves removing a portion of the patient's white blood cells using a process called leukapheresis. The T cells may then be expanded and mixed with expression vectors comprising the CAR polynucleotide in order to permanently transfer the CAR scaffold to the T cells. The T cells are expanded again and at the end of the expansion, the T cells are washed, concentrated, and then frozen to allow time for testing, shipping and storage until the patient is ready to receive the infusion of engineered T cells.

The term “co-administration” as used herein is meant either simultaneous administration or any manner of separate sequential administration of the immunomodulatory cell described herein, and a further active agent or agents, known to be useful in the treatment of cancer, including chemotherapy and radiation treatment. The term further active agent or agents, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered by injection and another compound may be administered orally.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10^{th} edition (Dec. 5, 2014), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule or anti-mitotic agents; platinum coordination complexes; alkylating agents; antibiotic agents; topoisomerase I inhibitors; topoisomerase II inhibitors; antimetabolites; hormones and hormonal analogues; signal transduction pathway inhibitors; non-receptor tyrosine kinase angiogenesis inhibitors; immunotherapeutic agents; proapoptotic agents; cell cycle signalling inhibitors; proteasome inhibitors; heat shock protein inhibitors; inhibitors of cancer metabolism; and cancer gene therapy agents.

### Methods

According to a further aspect of the invention, there is provided a method of engineering an immunomodulatory cell, comprising:

(a) providing an immunomodulatory cell;

(b) introducing the expression vector described herein into said immunomodulatory cell; and

(c) expressing said expression vector in the immunomodulatory cell.

As a non-limiting example, the CAR can be introduced as transgenes encoded by an expression vector as described herein. The expression vector can also contain a selection marker which provides for identification and/or selection of cells which received said vector.

Polypeptides may be synthesized in situ in the cell as a result of the introduction of polynucleotides encoding said CAR into the cell. Alternatively, said polypeptides could be produced outside the cell and then introduced thereto. Methods for introducing a polynucleotide construct into cells are known in the art and including, as non limiting examples, stable transformation methods wherein the polynucleotide construct is integrated into the genome of the cell or transient transformation methods wherein the polynucleotide construct is not integrated into the genome of the cell and virus mediated methods. Said polynucleotides may be introduced into a cell by, for example, recombinant viral vectors (e.g. retroviruses, adenoviruses), liposomes and the like. For example, transient transformation methods include for example microinjection, electroporation or particle bombardment. The polynucleotides may be included in vectors, more particularly plasmids or viruses, in view of being expressed in cells.

The terms “transfection”, “transformation” and “transduction” as used herein, may be used to describe the insertion of the expression vector into the target cell. Insertion of a vector is usually called transformation for bacterial cells and transfection for eukaryotic cells, although insertion of a viral vector may also be called transduction. The skilled person will also be aware of the different non-viral transfection methods commonly used, which include, but are not limited to, the use of physical methods (e.g. electroporation, cell squeezing, sonoporation, optical transfection, protoplast fusion, impalefection, magnetofection, gene gun or particle bombardment), chemical reagents (e.g. calcium phosphate, highly branched organic compounds or cationic polymers) or cationic lipids (e.g. lipofection). Many transfection methods require the contact of solutions of plasmid DNA to the cells, which are then grown and selected for a marker gene expression.

Once the CAR has been introduced into the immunomodulatory cell, said cell may be referred to as a “transformed immunomodulatory cell”. Therefore, in the scope of the present invention is also encompassed a cell line obtained from a transformed immunomodulatory cell according to the method previously described.

According to a further aspect of the invention, there is provided an engineered immunomodulatory cell comprising a chimeric antigen receptor (CAR) which binds to a protein on a target cell, wherein said CAR comprises:

a target binding domain,

a spacer domain which comprises at least one, or multiples of, domains 2, 3 or 4 or a combination thereof of a CD4 molecule,

a transmembrane domain and

an intracellular effector domain,

wherein the length of the spacer domain is such that the distance between the cell membranes of the target cell and engineered immunomodulatory cell creates an immune synapse.

The term “immune synapse” or “immunological synapse” refers to any stable, flattened interface between a lymphocyte or natural killer (NK) cell and a cell that they are in the process of recognising (as described in more detail in Huppa and Davis (2003) Nat. Immunol. 3, 973-983; Davis and van der Merwe (2006) Nat. Immunol. 7(8), 803-809; Rossy et al. (2012) Front. Immun. 3, 352, all of which are herein incorporated herein by reference in their entirety).

As explained herein, in natural CD8⁺ T cell and MHC Class I binding, an intercellular membrane contact region is formed which is defined in width by the physical dimensions of the TCR:antigen:MHC complex. Any inhibitory signals which are too large for this space are excluded which allows the TCR signals to activate cell killing. In one embodiment, the distance between the cells membranes is about 14 nm (or about 14 Å). This distance has been shown to be the dimensions of the natural TCR:peptide:MHC complex, therefore without being bound by theory, this is thought to be the optimum distance for creating an effective immune synapse.

### Examples

### Example 1: Construction of CAR (Chimeric Antigen Receptor) Containing Different Extracellular Linker Length

The generic CAR architecture investigated here comprises the target-specific scFv, variable length CD4 spacers (SEQ ID NOs: 2, 3 and 4), CD4 transmembrane domain (SEQ ID NO: 5), CD28 intracellular domain (SEQ ID NO: 6) and CD3zeta (CD3ζ) signaling domain (SEQ ID NO: 7).

The entire CAR construct is constructed allowing for the insertion of different CD4 spacer domains (SEQ ID NOs: 2, 3 and 4) as synthesised DNA-fragments by incorporating appropriate restriction sites in the CAR and DNA sequences. Standard molecular biology protocols are followed to PCR amplify, restriction enzyme digest, purify and ligate DNA fragments into expression vectors.

TABLE 1
Details of sequences used in CAR construct

| SEQ ID | | |
|---|---|---|
| NO. | Description | Sequence |
| 2 | CD4 Domain 4 | RATQLQKNLTCEVWGPTSPKLMLSLKL |
| | | ENKEAKVSKREKAVWVLNPEAGMWQCL |
| | | LSDSGQVLLESNIKVLP |
| 3 | CD4 Domain 3 | LAFQKASSIVYKKEGEQVEFSFPLAFT |
| | and Domain 4 | VEKLTGSGELWWQAERASSSKSWITFD |
| | | LKNKEVSVKRVTQDPKLQMGKKLPLHL |
| | | TLPQALPQYAGSGNLTLALEAKTGKLH |
| | | QEVNLVVMRATQLQKNLTCEVWGPTSP |
| | | KLMLSLKLENKEAKVSKREKAVWVLNP |
| | | EAGMWQCLLSDSGQVLLESNIKVLP |
| 4 | CD4 Domain 2, | FGLTANSDTHLLQGQSLTLTLESPPGS |
| | Domain 3, | SPSVQCRSPRGKNIQGGKTLSVSQLEL |
| | Domain 4 | QDSGTWTCTVLQNQKKVEFKIDIVVLA |
| | | FQKASSIVYKKEGEQVEFSFPLAFTVE |
| | | KLTGSGELWWQAERASSSKSWITFDLK |
| | | NKEVSVKRVTQDPKLQMGKKLPLHLTL |
| | | PQALPQYAGSGNLTLALEAKTGKLHQE |
| | | VNLVVMRATQLQKNLTCEVWGPTSPKL |
| | | MLSLKLENKEAKVSKREKAVWVLNPEA |
| | | GMWQCLLSDSGQVLLESNIKVLP |
| 5 | CD4 | TWSTPVQPMALIVLGGVAGLLLFIGLG |
| | transmembrane | IFFSVRS |
| | domain | |
| 6 | CD28 | RSKRSRLLHSDYMNMTPRRPGPTRKHY |
| | intracellular | QPYAPPRDFAAYRS |
| | domain | |
| 7 | CD3ζ | RVKFSRSADAPAYQQGQNQLYNELNLG |
| | signalling | RREEYDVLDKRRGRDPEMGGKPRRKNP |
| | domain | QEGLYNELQKDKMAEAYSEIGMKGERR |
| | | RGKGHDGLYQGLSTATKDTYDALHMQA |
| | | LPPR |

### Example 2: Confirmation of Antigen Binding of scFv Using SPR/BIAcore

Soluble scFv fragments produced and purified from mammalian expression systems are subjected to in vitro affinity determination to their antigen. A dilution series of scFv protein in HBS-EP buffer is injected over a BIAcore T200 chip surface previously coated with the antigen at an appropriate ‘Response Unit Density’ and the sensogram recorded. Analysis of the binding kinetics is assisted by the proprietary software using an appropriate fitting model (mostly 1:1 binding). Affinity data can be used to confirm suitability of scFv fragments to be used in the CAR construct.

### Example 3: Expression of CAR on T-Cells

In brief, host T cells are transfected or transduced with the appropriate CAR construct using standard protocols known in the art. Mammalian expression vectors may be used for transient cell surface expression or retroviral vector transduction may be used for stably inserted CARs.

### Example 4: Determination of Antigen Binding of a CAR when Expressed on a Cell Surface

Affinity of scFvs in the context of CARs expressed on T-cells are determined by a receptor binding assay. Here, the fraction of soluble antigen bound to the CAR is determined over a range of increasing concentrations. The fraction bound is measured using flow cytometry and plotted against the concentration used providing the IC_{50%} (inhibitory concentration). The cytometer values are normalised for receptor numbers on T-cells by using Bangs Beads (Bangs Laboratories, Inc., Fishers, Ind.) coated with an anti-scFv detection mAb following standard protocols. The results from this assay are used to provide confidence that the signalling/T-cell stimulation originates from specific antigen binding.

### Example 5: Functional Cell Assay of Target Binding in the Context of an ‘Immune Synapse’

The ability of the different CAR-T constructs after transduction of T-cells is measured by using a commercially available reporter cell line (Promega Immunostimulatory Bioassay T-cell activation bioassay (IL-2; cat# CS1870002 or NFAT, cat#CS176404)). Binding of the cell-surface displayed CAR to its antigen on another cell type will activate signalling through the CD3 and CD28 signalling pathway, respectively. The reporter cell line (i.e. Jurkat cells) has been re-engineered in such a way that the T-cell activation will result in luciferase transcription/translation via an IL-2 promoter or NFAT-RE.

CAR constructs with different length CD4 spacers are compared using the data obtained from the assays used in Examples 4 and 5 to determine the optimum spacer length to be used, with a specific target antigen, for T-cell activation from immune synapse formation with target displaying cells.

### Example 6: Generation of Target Specific CARs

Peripheral blood mononuclear cells (PBMCs) of healthy donors were obtained after centrifugation of fresh blood on a density gradient using Accuspin Sytem-Histopaque (Sigma, A7054) according to the manufacturer's instructions. Cells were then resuspended at 1×10⁶ cells/ml and cultured in 24-well plates in TexMAcs medium (Miltenyi Biotech; 130-097-196) containing 100 IU/ml of IL-2 (Sigma; SRP3085) and beads coated with specific antibodies for CD3 and CD28 (TransAct beads, Miltenyi Biotec) to initiate outgrowth of T cells.

48 hours post activation, T-cells were infected with lentivirus encoding CARs targeted to BCMA (αBCMA CARs). A multiplicity of infection (MOI) of 5 was used. 5 days post transduction, expression of CARs on the T cell surface was assayed by flow cytometry using antigen-Fc AlexaFluor 647 (produced and conjugated in-house; ThermoFisher; A20006) in combination with ZsGreen expression.

Fresh medium and IL-2 were added 3 times per week during culture and cell concentration maintained at about 0.7×10⁶ cells/mL. 12 days post transduction, CAR T-cells were harvested and effector function tested using assays described below.

### Cytotoxicity Assay Results

Cytotoxicity assay was evaluated by flow cytometry. The target negative and positive cell lines (in-house generated) were suspended in PBS at 1×10⁶ cells/mL and stained with fluorescent Cell Trace Far Red (0.1 μM, final concentration; ThermoFisher; C34564) and with Cell Trace Violet (0.1 μM, final concentration; ThermoFisher; C34557) respectively. Cells were incubated at room temperature for 30 minutes, protected from light. The cells were then washed twice in medium containing 10% of serum and suspended in 4×10⁵ cells/mL. Stained cell types were combined and 100 μl of the obtained solution added to untransduced (UT) control or CAR-transduced T-cells at a 1:1 effector to target ratio.

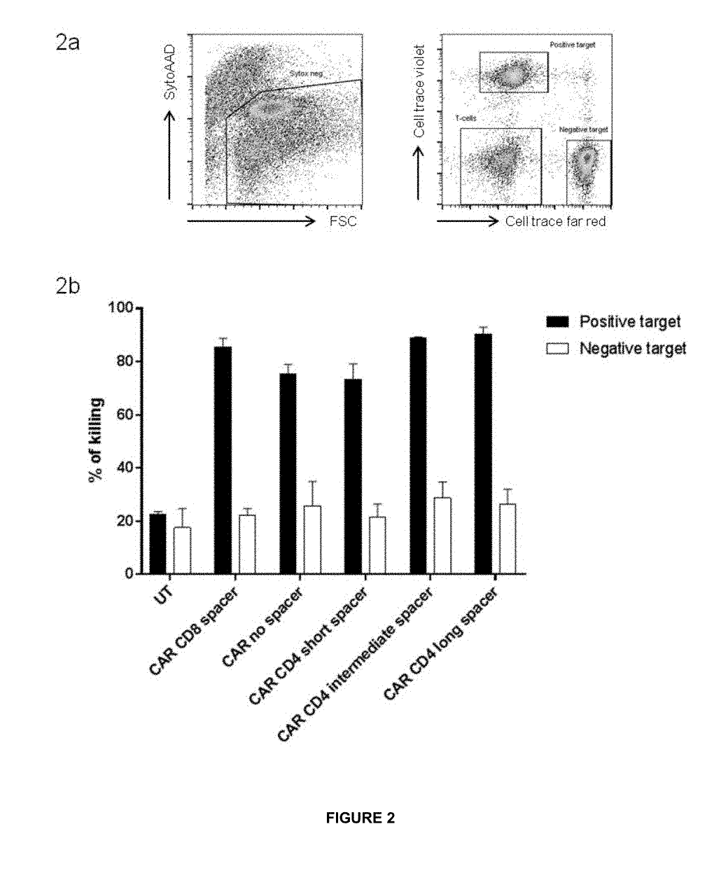

The cultures were incubated for 24 hours at 37° C. Immediately after the incubation, a solution containing SytoxAADavanced (20 μM, final concentration; ThermoFisher; S10349), EDTA (200 mM final concentration) and Dnase (10 mM final concentration) was added, incubated for 20 minutes and flow cytometry acquisition was performed. Samples were acquired using a MACSQuant flow cytometer (Miltenyi Biotec), and data analyzed using FlowJo. An example of the gating strategy is shown in FIG. 2A.

The percentage of survival of target cells was calculated as follows:

100−(sample counts/maximum counts)×100 where the maximum count is given by the number of target cells in the absence of any effector cells.

### Intracellular Cytokine Staining Assay (ICCS) Results:

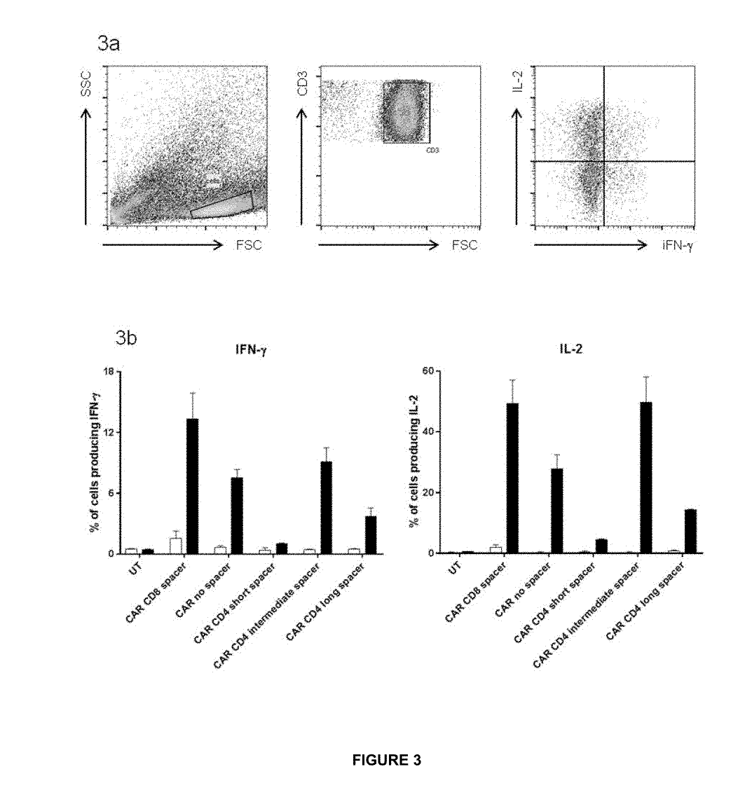

For intracellular cytokine staining, 2×10⁵ T-cells were cultured alone or in the presence of 2×10⁵ target cells (negative or positive target expressing cells as above). The samples were incubated at 37° C. for 6 hours, in the presence of Brefeldin A (BD, 555029). The cells were surface stained with anti-CD3 (BioLegend, clone UCHT1), then permeabilized, and intracellular staining was conducted for IFN-γ (BioLegend, clone 5S.B3) and IL-2 (BioLegend, clone MQ1-17H12) by following the instructions of the Cytofix/Cytoperm kit (Caltagmedsystem, GAS-002). Samples were acquired using a MACSQuant flow cytometer (Miltenyi Biotec), and data analyzed using FlowJo. An example of the gating strategy is shown in the FIG. 3A.

TABLE 2
Other relevant sequences

| SEQ ID | | |
|---|---|---|
| NO. | Description | Sequence |
| 1 | CD4 sequence | MNRGVPFRHLLLVLQLALLPAATQG |
| | (UniProt, ID | KKVVLGKKGDTVELTCTASQKKSIQ |
| | number P01730) | FHWKNSNQIKILGNQGSFLTKGPSK |
| | | LNDRADSRRSLWDQGNFPLIIKNLK |
| | | IEDSDTYICEVEDQKEEVQLLVFGL |
| | | TANSDTHLLQGQSLTLTLESPPGSS |
| | | PSVQCRSPRGKNIQGGKTLSVSQLE |
| | | LQDSGTWTCTVLQNQKKVEFKIDIV |
| | | VLAFQKASSIVYKKEGEQVEFSFPL |
| | | AFTVEKLTGSGELWWQAERASSSKS |
| | | WITFDLKNKEVSVKRVTQDPKLQMG |
| | | KKLPLHLTLPQALPQYAGSGNLTLA |
| | | LEAKTGKLHQEVNLVVMRATQLQKN |
| | | LTCEVWGPTSPKLMLSLKLENKEAK |
| | | VSKREKAVWVLNPEAGMWQCLLSDS |
| | | GQVLLESNIKVLPTWSTPVQPMALI |
| | | VLGGVAGLLLFIGLGIFFCVRCRHR |
| | | RRQAERMSQIKRLLSEKKTCQCPHR |
| | | FQKTCSPI |

1. A chimeric antigen receptor (CAR) comprising:
a target binding domain;
a spacer region;
a transmembrane domain; and
an intracellular effector domain,
wherein the spacer region comprises at least one, or multiples of, domains 2, 3 or 4 or a combination thereof of a CD4 molecule.

2. The chimeric antigen receptor of claim 1, wherein the spacer region comprises domain 4 of a CD4 molecule.

3. The chimeric antigen receptor of claim 1, wherein the spacer region comprises domains 3 and 4 of a CD4 molecule.

4. The chimeric antigen receptor of claim 1, wherein the spacer region comprises domains 2, 3 and 4 of a CD4 molecule.

5. The chimeric antigen receptor of claim 1, wherein the spacer region comprises domains 2 and 3 and two copies of domain 4 of a CD4 molecule.

6. The chimeric antigen receptor of claim 1, wherein domain 2 of a CD4 molecule comprises amino acids 126 to 203 of SEQ ID NO: 1.

7. The chimeric antigen receptor of claim 1, wherein domain 3 of a CD4 molecule comprises amino acids 204 to 317 of SEQ ID NO: 1.

8. The chimeric antigen receptor of claim 1, wherein domain 4 of a CD4 molecule comprises amino acids 318 to 374 of SEQ ID NO: 1.

9. The chimeric antigen receptor of claim 1, wherein the target binding domain comprises an antibody, an antigen binding fragment or a ligand.

10. The chimeric antigen receptor of claim 1, wherein the target binding domain binds to a tumour associated antigen.

11. The chimeric antigen receptor of claim 10, wherein the tumour associated antigen is selected from: BCMA, CD19, HER2, prostate stem cell antigen (PSCA), prostate-specific membrane antigen (PSMA), carcinoembryonic antigen (CEA), cancer antigen-125, CA19-9, MUC-1, tyrosinase, CD34, CD45, CD117, protein melan-A, synaptophysis, CD22, CD27, CD30, CD70, ganglioside G2 (GD2), epidermal growth factor variant III (EGFRvIII), mesothelin, prostatic acid phosphatise (PAP), prostein, TARP, Trp-p8 or six transmembrane epithelial antigen of the prostate I (STEAP1).

12. The chimeric antigen receptor of claim 1, wherein the target binding domain has a binding affinity of less than about 500 nanomolar (nM).

13. The chimeric antigen receptor of claim 1, wherein the transmembrane domain comprises the transmembrane domain of CD4.

14. The chimeric antigen receptor of claim 1, wherein the intracellular effector domain comprises a CD3zeta signalling domain.

15. The chimeric antigen receptor of claim 1, wherein the intracellular effector domain additionally comprises a costimulatory domain.

16. The chimeric antigen receptor of claim 15, wherein the costimulatory domain comprises the intracellular domain of a costimulatory molecule, selected from CD28, CD27, 4-1BB, OX40, ICOS, CD30, CD40, PD-1, CD2, CD7, LIGHT, NKG2C, B7-H3 or any combination thereof.

17. A polynucleotide encoding the chimeric antigen receptor of claim 1.

18. An expression vector comprising the polynucleotide of claim 17.

19. An immunomodulatory cell comprising the chimeric antigen receptor of claim 1.

20. The immunomodulatory cell of claim 19, which is derived from an inflammatory T-lymphocyte, cytotoxic T-lymphocyte, regulatory T-lymphocyte or helper T-lymphocyte.

21. A method of treating a patient in need thereof, comprising administering the immunomodulatory cell of claim 19.

22. A method of engineering an immunomodulatory cell, comprising:
(a) providing an immunomodulatory cell;
(b) introducing an expression vector comprising a polynucleotide encoding a chimeric antigen receptor (CAR) comprising:
a target binding domain;
a spacer region;
a transmembrane domain; and
an intracellular effector domain,
wherein the spacer region comprises at least one, or multiples of, domains 2, 3 or 4 or a combination thereof of a CD4 molecule into said immunomodulatory cell; and
(c) expressing said expression vector in the immunomodulatory cell.

23. An engineered immunomodulatory cell comprising a chimeric antigen receptor (CAR) which binds to a protein on a target cell, wherein said CAR comprises:
a target binding domain,
a spacer domain which comprises at least one, or multiples of, domains 2, 3 or 4 or a combination thereof of a CD4 molecule,
a transmembrane domain and
an intracellular effector domain,
wherein the length of the spacer domain is such that the distance between the cell membranes of the target cell and engineered immunomodulatory cell creates an immune synapse.

24. The immunomodulatory cell of claim 23, wherein the distance between the cells membranes is about 14 nm.


SEQUENCE LISTING (7 sequences)

SEQ ID NO: 1 - protein, 458 aa, Human
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 2 - protein, 71 aa, Human
RATQLQKNLTCEVWGPTSPKLMLSLKLENKEAKVSKREKAVWVLNPEAGMWQCLLSDSGQVLLESNIKVLP

SEQ ID NO: 3 - protein, 187 aa, Human
LAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLP

SEQ ID NO: 4 - protein, 266 aa, Human
FGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLP

SEQ ID NO: 5 - protein, 34 aa, Human
TWSTPVQPMALIVLGGVAGLLLFIGLGIFFSVRS

SEQ ID NO: 6 - protein, 41 aa, Human
RSKRSRLLHSDYMNMTPRRPGPTRKHYQPYAPPRDFAAYRS

SEQ ID NO: 7 - protein, 112 aa, Human
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR